(19) United States
(12) Patent Application Publication
(10) Pub. No.: US 2023/0030380 A1
(43) Pub. Date: Feb. 2, 2023

(54) METHOD AND APPARATUS FOR DETERMINING INTERNAL ORGAN SHIFT

(71) Applicant: Brainlab AG, Munich (DE)

(72) Inventors: Stefan VILSMEIER, Munich (DE); Claus Promberger, Munich (DE)

(21) Appl. No.: 17/783,675

(22) PCT Filed: Dec. 10, 2019
(86) PCT No.: PCT/EP2019/084349

§ 371 Date: Jun. 9, 2022

(51) Int. Cl. A61B 5/06; A61B 5/00

(52) U.S. Cl. CPC A61B 5/063; A61B 5/746; A61B 2560/0223

(57) ABSTRACT

Provided are a method and apparatus for determining displacement of an internal object disposed in a patient's body. The method includes positioning an arrangement of electrodes of a capacitive sensor adjacent to a patient's body part, such that each electrode is spaced apart from the body part, wherein the body part at least partly encloses the internal object of the patient. Providing, with the capacitive sensor, a plurality of sensor signals, wherein each sensor signal is indicative of a capacitance in a vicinity of at least one electrode of the arrangement of electrodes, determining a set of capacitance values for at least a subset of the electrodes of the arrangement of electrodes based on processing the plurality of sensor signals, and determining a displacement of the internal object with respect to the body part based on comparing the determined set of capacitance values with a set of reference capacitance values.

### FIELD OF THE INVENTION

The present invention relates to a method and an apparatus for determining a displacement and/or shift of an internal object of a patient, such as e.g. a displacement and/or shift of an internal organ. Further, the invention relates to the use of a capacitive sensor for determining a displacement of an internal object of a patient.

### TECHNICAL BACKGROUND

In many medical applications or procedures, such as for example surgical procedures and radiotherapy procedures, it may be desirable to determine a displacement of an internal object of a patient. For example, tissue to be removed, excised and/or resected in a surgical procedure or to be irradiated in a radiation treatment, such as e.g. cancerous tissue, is captured in a pre-operative image or scan of at least a part of the patient's body. During the actual surgery or radiation treatment, or in an individual treatment session of a series of consecutive treatment sessions, however, it may be desirable to have information on the position, location and/or orientation of the object to be treated, which might under certain circumstances have changed, e.g. with respect to the pre-operative scan and/or with respect to a preceding treatment session, due to a shift or displacement of the internal object. Such shift or displacement can, for instance, be caused by partial removal, excision and/or resection of the internal object and/or tissue surrounding or arranged in the vicinity of the internal object. Also, such shift or displacement may be caused by movements or displacements of soft tissue surrounding or constituting the internal object. Also, it may be desirable to track a position and/or location of the internal object e.g. during a medical treatment or a treatment session and/or in a series of treatment sessions.

In general, various approaches or tracking systems can be used to determine a displacement of an internal object. For example, markers that are visible in a camera can be used for optically tracking the internal object. Also, an electro-magnetic or magnetic field strength of markers attached to the patient can be detected for tracking the internal object and/or determining a displacement thereof. Other approaches or tracking systems can be based on analyzing video images of the patient and determining the displacement of the internal organ or object without the use of markers. If markers are used, they usually have to be attached to the patient and their position relative to the patient anatomy should be known. Also, X-ray, CT (Computed Tomography), MRT (Magnetic Resonance Tomography), ultrasound or other imaging modalities can be used to image the interior of the patient's body and to derive information on a shift or displacement of the internal object.

These conventional approaches and systems, however, have certain limitations and drawbacks, as changes in a position of an internal object are generally hard to determine and/or track. For example, the position of an internal object disposed in a patient's body can hardly be determined or tracked using optical methods, e.g. if a line of sight of the optical device or camera is blocked or obscured. The same applies to magnetic or electromagnetic tracking methods. Further, determining the displacement of the internal object and/or tracking the position thereof using imaging modalities for imaging the patient's interior are costly and time-consuming. Moreover, at least some of these techniques, such as X-ray or CT imaging, result in an additional radiation dose for the patient and hence should be used sparingly.

It is, therefore, desirable to provide for an improved method and apparatus for determining a displacement of an internal object of a patient relative to a body part of the patient, in which the above-mentioned drawbacks are at least mitigated or at least partly overcome.

The present invention can be used for medical procedures e.g. in connection with a system for image-guided radiotherapy such as ExacTrac or for image-guided cranial navigation, both products of Brainlab AG.

Aspects of the present disclosure, examples and exemplary steps or features and their embodiments are disclosed in the following. Different aspects, embodiments, examples and exemplary features of the present disclosure can be combined in accordance with the disclosure or invention wherever technically expedient and feasible.

### EXEMPLARY SHORT DESCRIPTION OF THE INVENTION

In the following, an exemplary short description of some of the steps and/or features of the present disclosure is given which shall not be understood to limit the invention or disclosure only to the features and steps or a combination thereof described in this section.

According to the present disclosure, a method for determining a displacement of an internal object disposed in a body of a patient as well as a corresponding apparatus are provided. The method comprises positioning an arrangement of electrodes of a capacitive sensor (also referred to as capacitance sensor) adjacent to a body part of a patient, such that each electrode is spaced apart from the body part. Therein, the internal object may refer to any structure and/or anatomic structure of the patient that is arranged partly or entirely in an interior volume of the patient. For example, the internal object may refer to at least a part of an internal organ of the patient, at least a part of a brain of the patient, at least a part of a lung of the patient, at least a part of a liver of the patient, at least a part of a spine of the patient or any other internal structure or object.

Further, the method comprises the step of providing, with the capacitive sensor, a plurality of sensor signals, wherein each sensor signal is indicative of a capacitance in a vicinity of at least one electrode of the arrangement of electrodes. Therein, the capacitance in the vicinity of the at least one electrode can, for example, be a capacitance between said at least one electrode and a ground or ground potential, between said at least one electrode and an outer surface of the patient, between said at least one electrode and a reference electrode of the capacitance sensor, and/or between said at least one electrode and a further electrode of the capacitance sensor.

Moreover, the method comprises the steps of determining a set of capacitance values for at least a subset of the electrodes of the arrangement of electrodes based on processing the plurality of sensor signals, and determining a displacement of the internal object with respect to the body part based on comparing the determined set of capacitance values with a set of reference capacitance values. Generally, the displacement of the internal object may be referred to as internal organ shift in the context of the present disclosure. Further, the displacement with respect to the body part may, for instance, refer to a displacement and/or shift of the internal object with respect and/or relative to tissue of the patient that at least partly surrounds the internal object.

The present invention, generally, allows to precisely, accurately and reliably determine the internal organ shift based on, by means of and/or using a capacitive sensor with an arrangement of electrodes in a non-invasive manner. Also, the displacement of the internal object can be determined in a contactless manner, which may mean that no or substantially no component of the device or apparatus used to detect the displacement may be in contact and/or may be touching the patient during determining the displacement. In particular, no markers may have to be attached to the patient, which can increase a comfort for the patient as well as simplify and/or shorten a pre-operative procedure, e.g. for preparing the patient and/or an operating theatre for the actual medical treatment. Also, detecting the internal object's displacement in a contactless manner can increase a hygiene as well as simplify and/or shorten a post-operative procedure, such as e.g. a cleaning procedure for cleaning the operating theatre. Apart from that, according information about the displacement of the internal object can be provided e.g. to a surgeon or operator during the actual medical procedure. Hence, an overall workflow or medical workflow of the medical treatment can be improved, in particular because the internal object's displacement can be determined in a cost-efficient and time-efficient manner.

In addition, exposure of the patient to radiation for determining the displacement of the internal object can be avoided, thereby reducing an overall dose delivered to the patient and increasing a safety for the patient.

### GENERAL DESCRIPTION OF THE INVENTION

In this section, a description of the general features and/or steps of the present invention or disclosure is given for example by referring to possible embodiments of the invention or disclosure. The invention, however, is defined in the independent claims, wherein further embodiments are incorporated in the dependent claims as well as the foregoing and following description.

As stated above, it may be desirable to provide for an improved method and apparatus for determining a displacement of an internal object of a patient, in which the above-mentioned drawbacks of conventional or currently used approaches or systems are at least mitigated or at least partly overcome.

This is achieved by the subject matter of the independent claims, wherein further embodiments are incorporated in the dependent claims and the following description.

According to a first aspect of the present disclosure, there is provided a method for determining and/or detecting a displacement of an internal object disposed and/or arranged partly or entirely in a body of a patient. The method comprises at least the following steps:
positioning and/or arranging an arrangement of electrodes of a capacitive sensor adjacent to, near to and/or close to a body part of a patient, such that each electrode is spaced apart from the body part and/or such that each electrode is not touching the patient, wherein the body part at least partly encloses and/or surrounds the internal object of the patient; 1vproviding, generating and/or outputting, with the capacitive sensor, a plurality of sensor signals, wherein each sensor signal is indicative, representative and/or descriptive of a capacitance in a vicinity of at least one electrode of the arrangement of electrodes; 1

determining a set of capacitance values for at least a subset of, some of, and/or a part of the electrodes of the arrangement of electrodes based on processing some or all of the plurality of sensor signals; and
determining and/or detecting a displacement of the internal object with respect to and/or relative to the body part based on and/or by comparing the determined set of capacitance values with a set of reference capacitance values.

Generally, “the internal object” can refer to any structure, anatomic structure, anatomy, and/or anatomic feature arranged, disposed and/or located partly or entirely in an interior and/or interior volume of the patient. Accordingly, the internal object or at least a part thereof may not be visible from an outside of the patient (e.g. using optical means). For example, the internal object may refer to at least a part of an internal organ of the patient, at least a part of a brain of the patient, at least a part of a lung of the patient, at least a part of a liver of the patient, at least a part of a spine of the patient or any other internal structure or object. For example, the internal object can refer to, comprise and/or be arranged near cancerous tissue, e.g. which is to be excised, resected and/or removed from the patient's body in a surgical treatment of the patient and/or which is to be irradiated in radiation therapy.

In the context of the present disclosure, the “displacement of the internal object”, can refer to and/or comprise a shift, a change in position, a change in location, and/or a change in orientation of the internal object. Generally, the displacement of the internal object can be given in absolute or relative values. Further, the displacement of the internal object can be referred to as displacement of an internal organ and/or as internal organ shift in the context of the present disclosure. It should be noted, however, that the object's displacement “with respect to the body part” may mean that the object is displaced locally and/or internally, e.g. in the patient's interior volume and/or within the patient's body. Accordingly, the displacement of the internal object with respect and/or relative to the body part may not be equated with a global movement of the patient, i.e. a patient motion, which may also referred to hereinafter as global displacement of the internal object. In other words, the displacement of the internal object relative to the body part may refer to a displacement of the object in a patient's coordinate system. Such local displacement of the internal object, however, can e.g. be determined with respect to a reference position, location and/or orientation of the internal object, for example a previous position, previous location and/or previous orientation of the internal object, e.g. a position, location and/or orientation as described and/or indicated by the set of reference capacitance values.

In certain medical applications, treatments and/or procedures, such as for example surgical procedures and/or radiotherapy procedures, it may be desirable to determine and/or detect a displacement of an internal object and/or an internal organ shift. For example, it may be desirable to have information about a position, a positional change, a location, a change in location, an orientation and/or a change in orientation of the internal object, which is to be treated in a medical application, for example, which is to be at least partly removed and/or resected in a surgical procedure and/or which is to be irradiated in a radiation treatment. During the actual surgery or radiation treatment, or in an individual treatment session of a series of consecutive treatment sessions, however, the position, location and/or orientation of the internal object may have changed with respect to a previous position, location and/or orientation thereof, e.g. with respect to a position, location and/or orientation of the object in a pre-operative scan and/or with respect to a position, location and/or orientation of the object in a preceding treatment session. Such change in position, location and/or orientation can be caused, for instance, by partial removal, excision and/or resection of the internal object and/or tissue surrounding or being arranged close to the internal object. Also, such displacement or shift of the internal object can be caused by movements of soft tissue surrounding and/or constituting the internal object.

The present invention advantageously allows to determine the displacement of the internal object and/or the internal organ shift in a precise, accurate, reliable, fast, efficient and non-invasive manner. Also, the displacement of the internal object can be determined in a contactless manner, which may mean that no or substantially no component of the device or apparatus used to detect the displacement may be in contact with and/or may be touching the patient during determining the displacement. In particular, no markers may have to be attached to the patient, which can increase a comfort for the patient as well as simplify and/or shorten a pre-operative procedure, e.g. for preparing the patient and/or an operating theatre for the actual medical treatment. Also, detecting the internal object's displacement in a contactless manner can increase a hygiene as well as simplify and/or shorten a post-operative procedure, such as e.g. a cleaning procedure for cleaning the operating theatre. Hence, an overall workflow or medical workflow of the medical treatment can be improved by the present invention, in particular because the internal object's displacement can be determined in a cost-efficient and time-efficient manner. In addition, exposure of the patient to radiation for determining the displacement of the internal object can be avoided, thereby reducing an overall dose delivered to the patient, and thereby increasing a safety for the patient.

It should be noted that the step of determining the displacement of the internal object relative to the body part is to be understood, interpreted and/or construed in a broad manner. Accordingly, determining the displacement may comprise determining an occurrence of the displacement. In other words, determining the displacement may comprise determining whether or not the internal object has been displaced relative to the body part, e.g. with respect to a reference position, location and/or orientation of the internal object. Alternatively or additionally, the step of determining the displacement of the internal object may comprise determining an extent, a size and/or an amount of the displacement, e.g. with respect to a reference position, location and/or orientation of the internal object. In other words, determining the displacement may comprise determining a distance by which the internal object has been displaced relative to the body part. Alternatively or additionally, determining the displacement may comprise determining a direction, in which the internal object has been displaced, e.g. with respect to a reference position, location and/or orientation of the internal object. Alternatively or additionally, determining the displacement may comprise determining occurrence of a change in orientation of the internal object and/or determining a change in orientation of the internal object, e.g. with respect to a reference position, location and/or orientation of the internal object. Moreover, the displacement of the internal object may be determined in two-dimensional or three-dimensional space. The reference position, location and/or orientation of the internal object may, for instance, be described, represented and/or indicated by the set of reference capacitance values.

The term “arrangement of electrodes” is to be understood, interpreted and/or construed in a broad manner. Accordingly, the arrangement of electrodes may refer to an arbitrary structural configuration of a plurality of electrodes, such as an arrangement of the electrodes in a regular pattern, a non-regular pattern, a uniform pattern, or a non-uniform pattern. For instance, at least some of or all of the electrodes of the arrangement of electrodes can be positioned in a grid, an array, a matrix, a row, a line, or any combination thereof.

Further, the step of providing the plurality of sensor signals may comprise driving the capacitive sensor and/or supplying the capacitive sensor with electrical energy in order to provide the sensor signals.

Moreover, the sensor signals may e.g. be provided to one or more processors, a processing circuitry and/or a control circuitry. Therein, the at least one processor, the processing circuitry and/or the control circuitry may be part of an apparatus for determining the object's displacement and/or of the capacitive sensor.

Further, the set of capacitance values may be determined by the one or more processors, the processing circuitry and/or the control circuitry based on and/or by processing some or all of the sensor signals. Therein, the step of determining the set of capacitance values may comprise deriving the set of capacitance values from the sensor signals or at least a part thereof. Further, the set of capacitance values may refer to a plurality of capacitance values for the capacitances of the at least subset of electrodes. It emphasized that the set of capacitance values can be determined based on processing some of or all of the sensor signals of the capacitive sensor.

In the following, the method according to the first aspect is re-phrased. An arrangement of electrodes of a capacitive sensor can be positioned and/or located close to a body part of the patient, which body part at least partly surrounds the internal object. Therein, the actual positioning can optionally be performed in an at least partly automated manner, e.g. using a respective positioning device. Further, all or some of the electrodes of the arrangement of electrodes can be electrically driven and/or controlled, such that a plurality of sensor signals is generated by the capacitive sensor. Therein, each of the sensor signals may contain information about the capacitance in a vicinity of, close to and/or near one or more of the electrodes of the arrangement of electrodes. The sensor signals may then be processed, e.g. by at least one processor, a processing circuitry and/or a control circuitry, and a plurality of capacitance values of the capacitances of at least a part and/or subset of the electrodes can be derived therefrom. Further, the set of determined capacitance values can be compared to the set of reference capacitance values. Based on this comparison, the displacement of the internal object can be determined and/or detected, as the capacitance and/or the value of the capacitance in the vicinity of an electrode depends on, is altered, modified and/or changed by the material in the vicinity of said electrode.

According to an embodiment, the step of determining the displacement of the internal object comprises determining an occurrence of the displacement of the internal object with respect to the body part, and determining at least one of a size and a direction of the displacement of the internal object with respect to the body part. Accordingly, it may be determined whether or not a displacement of the internal object has occurred based on and/or by comparing the determined set of capacitance values with the set of reference capacitance values. In addition, one or both of the size and the direction of the displacement may be determined. Therein, the size of the displacement may refer to an amount of the displacement and/or a distance, by which the internal object has been displaced and/or moved within the interior volume of the patient. Further, the direction of the displacement may refer to a spatial direction in an arbitrary coordinate system, such as e.g. the patient's coordinate system, a coordinate system of the apparatus used for determining the displacement, a coordinate system of an operating theatre, a coordinate system of a medical apparatus or system for performing a medical treatment on the patient or any other coordinate system. Generally, determining occurrence of the displacement and one or both of the size and direction of the displacement may allow to precisely determine a position, orientation and/or location of the internal object after the displacement has occurred. In turn, this information can be used for the medical treatment, e.g. for resecting at least a part of the object and/or for irradiating at least a part of the object, thereby increasing an overall efficiency of the treatment.

According to an embodiment, each sensor signal and/or each capacitance value is indicative, descriptive and/or representative of at least one of a capacitance between one of the electrodes and a ground (or ground potential), between one of the electrodes and an outer surface of the patient, e.g. an outer surface facing and/or being arranged opposite to the arrangement of electrodes, between one of the electrodes and a reference electrode of the capacitance sensor, and between two electrodes of the capacitance sensor. Alternatively or additionally, the capacitance in the vicinity of the at least one electrode of the arrangement of electrodes is at least one of a capacitance between said at least one electrode and a ground (or ground potential), between said at least one electrode and an outer surface of the patient, e.g. an outer surface facing and/or being arranged opposite to the arrangement of electrodes, between said at least one electrode and a reference electrode of the capacitance sensor, and between said at least one electrode and a further electrode of the capacitance sensor.

Further, the capacitance value of an electrode of the arrangement of electrodes may be determined based on applying a known charge to said electrode and measuring a potential, by applying a known potential to said electrode and measuring a charge of the electrode (e.g. based on integrating the current supplied to the electrode over time), by constructing an oscillating circuit comprising said electrode, whereby the frequency of that circuit may depend on the capacitance value, and measuring the frequency. In another example implementation, a capacitance bridge may be employed to measure the capacitance value of said electrode.

Moreover, the set of capacitance values may be determined based on sequentially interrogating the electrodes of the arrangement of electrodes. For example, when a given electrode is not being interrogated, it could be grounded. Alternatively, the set of capacitance values may be determined based on simultaneously interrogating at least a subset of, some of or all of the electrodes of the arrangement of electrodes.

According to an embodiment, the set of reference capacitance values is indicative, representative and/or descriptive of the capacitance values of the at least subset of electrodes when the arrangement of electrodes is positioned at a reference position and/or in a reference orientation with respect to the body part and/or with respect to the internal object. Therein, the set of reference capacitance values can be determined in a measurement and/or calibration measurement, e.g. a measurement on the patient or using a phantom. Alternatively or additionally, the set of reference capacitance values can be determined in a simulation calculation, e.g. allowing to determine the expected capacitance values and/or the expected sensor signals, when the arrangement of electrodes is positioned at the reference position and/or in the reference orientation.

According to an embodiment, the set of capacitance values is determined at a measurement time. Alternatively or additionally, the set of reference capacitance values is indicative, representative and/or descriptive of the capacitance values of the at least subset of electrodes at a reference time preceding the measurement time. Therein, the measurement time can refer to a measurement time period or a measurement time instant. Likewise, the reference time can refer to a reference time period or a reference time instant.

According to an embodiment, the method further comprises determining the set of reference capacitance values based on positioning, e.g. at a reference time, the arrangement of electrodes at a reference position and in a reference orientation with respect to the body part of the patient and based on measuring the reference capacitance values using the capacitive sensor. Such measurement may be considered as calibration measurement and/or may be carried out on the actual patient or on a phantom resembling at least a part of the patient, e.g. resembling at least the body part. For example, the phantom can be at least partly filled with water to resemble the at least part and/or the body part of the patient. By measuring the set of reference capacitance values, the displacement of the internal object can reliably and accurately be determined.

The actual measurement of the set of reference capacitance values can, for example, be determined before carrying out the actual medical treatment. Alternatively, the set of reference capacitance values can be determined during the actual medical treatment. Also, the set of reference capacitance values can be determined multiple times during the medical treatment. For instance, an operator or user can trigger measurement of the set of reference capacitance values, e.g. based on or by actuating a switch, button, a user interface or any other actuating element of an apparatus for determining the internal object's displacement.

According to an embodiment, the method further comprises determining the set of reference capacitance values based on a simulation calculation with the arrangement of electrodes being positioned at a reference position and in a reference orientation with respect to the body part of the patient. Such simulation calculation may, for example, be based on a finite-element-method or any other suitable simulation method or technique.

According to an embodiment, the method further comprises storing the determined set of reference capacitance values in a data storage. By storing the reference capacitance values in the data storage, these values may be accessed, retrieved and/or processed quickly during the actual determination of the internal object's displacement and/or during the medical treatment.

According to an embodiment, the method further comprises and/or the step of determining the set of reference capacitance values comprises registering the capacitive sensor, the arrangement of electrodes and/or at least a part or subset of the electrodes with respect to and/or against a position of the body part of the patient. In other words, the method and/or the step of determining the set of reference capacitance values may comprise referencing the capacitive sensor, the arrangement of electrodes and/or at least a part or subset of the electrodes with a relative position and/or orientation of the body part of the patient and the arrangement of electrodes (and/or the electrodes thereof). Based thereon, a spatial location of each point of the body part and/or the internal object within a space can be assigned to and/or associated with at least one of the electrodes. Accordingly, registering the capacitive sensor and/or the arrangement of electrodes may comprise determining the position of the arrangement of electrodes, of at least a subset of electrodes and/or of each electrode with respect to the patient, the patient's anatomy, the body part and/or the internal object. In turn, this allows to determine the position, location and/or orientation of the internal object and/or the body part in space based on processing the sensor signals of the capacitive sensor. Generally, the step of registering may be considered as and/or may comprise mapping the capacitive sensor and/or the arrangement of electrodes (and/or the electrodes thereof) to the patient, the body part of the patient and/or the internal object (or vice versa).

For the actual registration, a reference point or area on the patient may be selected and mapped to the arrangement of electrodes (or vice versa). For example, a reference marker, e.g. comprising dielectric material, may be attached at a known position to the patient and the sensor signals of the capacitance sensor may be processed to determine which of the electrodes detect or “see” the reference marker.

According to an embodiment, determining the displacement of the internal object comprises:
computing, determining and/or calculating a deviation and/or difference between each capacitance value of the determined set of capacitance values and at least one reference capacitance value of the set of reference capacitance values; and
comparing the computed, determined and/or calculated deviation and/or difference for each capacitance value of the determined set of capacitance values with a threshold value for the deviation and/or difference, thereby determining whether the capacitance in the vicinity of one or more electrodes has changed.

In other words, the deviations and/or differences between the set of capacitance values and the set of reference capacitance values can be determined elementwise and/or electrode-wise, i.e. for each element and/or value of the set of capacitance values and the corresponding element and/or value of the set of reference capacitance values. The determined deviations and/or differences may then be compared to one or more threshold values in order to determine whether or not the capacitance in the vicinity of one or more electrodes has changed. Optionally, it may be determined for which and/or for how many of the electrodes of the arrangement of electrodes the capacitance has changed. Further, the position or location of the electrodes, for which the capacitance has changed, among the arrangement of electrodes may optionally be determined.

According to an embodiment, the method further comprises identifying one or more electrodes in the vicinity of which the capacitance has changed, and:
determining occurrence of the displacement of the internal object with respect to the body part based on determining that the capacitance in the vicinity of one or more individual electrodes has changed; and/or
checking and/or determining an occurrence of a patient motion and/or a displacement of the body part relative to the arrangement of electrodes based on determining that the capacitance in the vicinity of at least one electrode arranged at a first boundary, edge and/or border of the arrangement of electrodes and in the vicinity of at least one further electrode arranged at a second boundary, edge and/or border opposite to the first boundary of the arrangement of electrodes has changed.

Therein, identifying the at least one electrode, in the vicinity of which the capacitance has changed, may comprise identifying and/or determining a position of said at least one electrode among the electrodes of the arrangement of electrodes. Further, if only the capacitance(s) in the vicinity of one or more individual electrodes has changed, e.g. at least one individual electrode arranged at a certain location or area of the arrangement of electrodes, such change may indicate that a local displacement of the internal object relative to the body part has occurred and/or that no patient motion relative to the arrangement of electrodes has occurred, wherein the latter may be referred to as global displacement of the internal object and/or the body part. Further, the change in capacitance of at least one individual electrode may mean that the changes in capacitance of the at least one electrode is asymmetric among the arrangement of electrodes, e.g. asymmetric with respect to a middle axis and/or any axis of symmetry of the arrangement of electrodes. Moreover, if the capacitances in the vicinity of one or more electrodes arranged at opposite boundaries and/or edges of the arrangement of electrodes have changed, such change may be caused by a movement and/or displacement of at least the body part of the patient and/or the entire patient relative to the arrangement of electrodes.

Accordingly, occurrence of the displacement of the internal object relative to the body part may be determined based on determining a degree of symmetry, a symmetry, a degree of asymmetry and/or an asymmetry of a pattern of changes in the capacitances in the vicinity of one or more electrodes among the arrangement of electrodes. Generally, this may allow to efficiently and reliably differentiate between a local displacement of the internal object, i.e. a displacement of the object relative to the body part, and a global displacement of the internal object, i.e. a displacement of at least the body part relative to the arrangement of electrodes.

According to an embodiment, the occurrence of the patient motion and/or displacement of the body part relative to the arrangement of electrodes is checked and/or determined based on:
determining a first number of electrodes arranged at the first boundary, edge and/or border of the arrangement of electrodes, in the vicinity of which the capacitance has changed;
determining a second number of electrodes arranged at the second boundary, edge and/or border of the arrangement of electrodes, in the vicinity of which the capacitance has changed; and
comparing the first number of electrodes arranged at the first boundary, edge and/or border with the second number of electrodes arranged at the second boundary, edge and/or border of the arrangement of electrodes.

Based on determining the first number and the second number of electrodes, a degree of symmetry, a symmetry, a degree of asymmetry and/or an asymmetry of a pattern of changes in the capacitances in the vicinity of one or more electrodes among the arrangement of electrodes can be determined. This allows reliably and accurately differentiating between a local displacement of the internal object and a global displacement of the internal object.

According to an embodiment, the occurrence of the patient motion and/or the displacement of the body part relative to the arrangement of electrodes is checked and/or determined based on determining that the first number of electrodes arranged at the first boundary, edge and/or border substantially equals and/or matches the second number of electrodes arranged at the second boundary, edge and/or border of the arrangement of electrodes. Therein, substantially equaling and/or matching may mean that at least 75% of the electrodes, in the vicinity of which the capacitances have changed, are arranged symmetrically with respect to a middle axis and/or any axis of symmetry of the arrangement of electrodes.

According to an embodiment, the threshold values for the deviations between the capacitance values and the reference capacitance values are determined based on a calibration measurement and/or based on a simulation calculation. Therein, the calibration measurement may be performed on the patient and/or using a phantom. For instance, capacitance values from a plurality of phantoms, e.g. selectively filled with water, air and/or a combination thereof, may be used. This may allow identifying and/or finding correspondence between the position of water and/or air near the arrangement of electrodes and the measured capacitance values and/or their expected changes, e.g. caused by varying the water and/or air content or filling of the phantom. According to an embodiment, the displacement of the internal object with respect to the body part is determined using a classificator, a classifier, a classificator circuitry, a classifier circuitry, an artificial intelligence module, an artificial neural network and/or a neural network. Using any such device may allow to reliably and quickly evaluate a measured pattern of capacitance values and/or changes thereof, e.g. in terms of whether or not a displacement of the internal object has occurred.

For training the classificator, the classifier, the classificator circuitry, the classifier circuitry, the artificial intelligence module, the artificial neural network and/or the neural network, changes in the capacitances in the vicinity of one or more electrodes may be detected and, based on e.g. MRT, CT, X-ray or any other image of the patient's internal structure or anatomy, these changes may be labelled for training purposes. For instance, a labelling may be performed in terms of whether or not a displacement of the internal object relative to the body part has occurred. Optionally, however, labelling can also be done including the extent and/or direction and/or type of an organ movement and/or object displacement.

According to an embodiment, the method further comprises:
determining, with at least one surface scanner, a first position of at least a part of a skin of the patient at a first time;
determining, with the at least one surface scanner, a second position of the at least part of the skin at a second time different than the first time; and
determining an occurrence of the displacement of the internal object with respect to the body part based on comparing the determined first position and the determined second position of the at least part of the skin.

Using and/or by means of the surface scanner, it can be determined if the patient has moved and/or if a patient motion has occurred. In turn, this allows reliably differentiating between a local or a global displacement of the internal object.

According to an embodiment, the method further comprises:
determining, with at least one distance sensor, a first distance between at least a part of the patient and at least one fixed point in an environment of the patient at a first time;
determining, with the at least one distance sensor, a second distance between the at least part of the patient and the at least one fixed point in the environment of the patient at a second time different than the first time; and
determining occurrence of the displacement of the internal object with respect to the body part based on comparing the determined first distance and the determined second distance between the at least part of the patient and the at least one fixed point.

Similar to the surface scanner, also using and/or by means of the distance scanner, it can be determined if the patient has moved and/or if a patient motion has occurred. In turn, this allows reliably differentiating between a local or a global displacement of the internal object.

According to an embodiment, the at least one surface scanner and/or the at least one distance sensor is at least one of a camera, a surface camera, a thermo-camera, a 3D camera, a stereo camera, a range camera, a laser sensor, a LIDAR sensor, a radar sensor, a time-of-flight sensor, and an ultrasound sensor.

According to an embodiment, the method further comprises generating an alert signal in response to determining the displacement of the internal object with respect to the body part. Generally, the alert signal can be any suitable signal, such as e.g. an acoustic signal, an optical signal and/or an audio-visual signal. For example, a color may be displayed on a user interface if a displacement of the internal object relative to the body part has been detected, e.g. in order to assist a surgeon or operator during a medical treatment.

According to an embodiment, the method further comprises:
providing a pre-operative scan of at least a part of the body part, the pre-operative scan including at least a part of the internal object; and
adjusting, modifying and/or adapting at least one of a shape, a geometry, a position and an orientation of the at least part of the internal object in the pre-operative scan based on the determined displacement of the internal object with respect to the body part.

Accordingly, based on the detected displacement of the internal object, the pre-operative scan can be augmented, modified, adjusted and/or adapted, such that the modified pre-operative scan provides information about a current location of the internal object. Generally, this may provide a comprehensive assistance to a surgeon during a medical treatment. Also, effectiveness of the medical treatment as well as safety for the patient can be increased.

According to an embodiment, the internal object is at least one of at least a part of an internal organ of the patient, at least a part of a brain of the patient, at least a part of a lung of the patient, at least a part of a liver of the patient, and at least a part of a spine of the patient. The present invention, however, is not limited to these types of internal objects. Rather, the present invention may likewise be used for determining a displacement of any other type of internal object, particularly any type of internal organ of a patient.

It should be noted that the method according to the first aspect of the present disclosure does not involve or in particular comprise or encompass any invasive step, which would represent a substantial physical interference with the body requiring professional medical expertise to be carried out and entailing a substantial health risk even when carried out with the required professional care and expertise. Particularly, the invention does not involve or in particular comprise or encompass any surgical or therapeutic activity. The invention is instead directed as applicable to the mere determination of a displacement of an internal object. For this reason alone, no surgical or therapeutic activity and in particular no surgical or therapeutic step is necessitated or implied by carrying out the method according to the first aspect.

Further, it should be noted that the method according to the first aspect of the disclosure is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

In a second aspect, the present disclosure is directed to a computer program which, when running on at least one processor (for example, a processor) of at least one computer (for example, a computer) or when loaded into at least one memory (for example, a memory) of at least one computer (for example, a computer), causes the at least one computer to perform the above-described method according to the first aspect. The present disclosure may alternatively or additionally relate to a (physical, for example electrical, for example technically generated) signal wave, for example a digital signal wave, carrying information which represents the program, for example the aforementioned program, which for example comprises code means which are adapted to perform any or all of the steps of the method according to the first aspect. A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, for example the internet. The invention according to the second aspect therefore may alternatively or additionally relate to a data stream representative of the aforementioned program.

In a third aspect, the present disclosure is directed to a non-transitory computer-readable program storage medium on which the program according to the second aspect is stored.

In a fourth aspect, the present disclosure is directed to at least one computer (for example, a computer), comprising at least one processor (for example, a processor) and at least one memory (for example, a memory), wherein the program according to the third aspect is running on the processor or is loaded into the memory, or wherein the at least one computer comprises the computer-readable program storage medium according to the third aspect.

According to a fifth aspect, the present disclosure relates to a use of a capacitive sensor including an arrangement of electrodes, as described hereinabove and hereinbelow, for determining a displacement of an internal object disposed partly or entirely in a body of a patient. The capacitive sensor including the arrangement of electrodes may be part of the apparatus for determining the internal object's displacement, as described hereinabove and hereinbelow.

Also, the present disclosure may relate to a use of an apparatus, as described hereinabove and hereinbelow, for determining a displacement of an internal object disposed partly or entirely in a body of a patient.

According to a sixth aspect of the present disclosure, there is provided an apparatus for determining a displacement of an internal object disposed partly or entirely in a body of a patient. The apparatus comprises a capacitive sensor including at least one arrangement of electrodes, wherein the at least one arrangement of electrodes is configured to be arranged adjacent to, close to and/or near a body part of a patient, which body part at least partly encloses the internal object. Further, the capacitive sensor is configured to generate a plurality of sensor signals, each sensor signal being indicative of a capacitance in a vicinity of at least one electrode of the arrangement of electrodes. The apparatus further comprises at least one processor, a processing circuitry and/or a control circuitry configured to receive and process the plurality of sensor signals to determine a set of capacitance values for at least a subset of the electrodes of the arrangement of electrodes. Therein, the at least one processor, the processing circuitry and/or the control circuitry is configured to determine a displacement of the internal object with respect to and/or relative to the body part based on comparing the determined set of capacitance values with a set of reference capacitance values.

Generally, the apparatus may be configured to perform any or all of the steps of the method for determining the displacement of the internal object according to the first aspect of the disclosure. Vice versa, the method according to the first aspect may be performed using the apparatus according to the sixth aspect of the present disclosure.

According to an embodiment, the arrangement of electrodes includes an array of electrodes and/or a grid of electrodes. Therein, the array and/or the grid of electrodes may be regular, uniform, non-regular and/or non-uniform. In other words, the electrodes may be arranged in a regular, uniform, non-regular and/or non-uniform pattern in the array and/or grid of electrodes.

According to an embodiment, the electrodes of the arrangement of electrodes are arranged in a uniform pattern. Accordingly, the electrodes of the arrangement electrodes may be equally spaced apart from each other.

According to an embodiment, the electrodes of the arrangement of electrodes are arranged in a three-dimensional configuration. Using a three-dimensional configuration may allow to infer and/or derive three-dimensional information and/or information in three-dimensional space from the sensor signals and/or the determined set of capacitance values. Accordingly, the displacement of the internal object may be determined in three-dimensional space.

According to an embodiment, the electrodes of the arrangement of electrodes are arranged in at least one of a semi-spherical configuration, a spherical configuration and an arc-shaped configuration. Such configurations may arc-like bridge over and/or at least partly encompass at least a part of the patient, such as e.g. at least a part of the body part, in which the internal object is disposed. This may allow determining the displacement in three-dimensional space.

According to an embodiment, the arrangement of electrodes includes an arc-shaped array of electrodes and at least one further arc-shaped array of electrodes, wherein the arc-shaped array and the at least one further arc-shaped array are directed and/or extend in different directions and/or spatial directions. Also such configuration may advantageously be used for determining the displacement of the internal object in three-dimensional space.

According to an embodiment, the electrodes of the arrangement of electrodes are arranged on at least one of a head clamp for immobilizing a head of the patient, an immobilization mask for immobilizing at least a part of the patient, and a patient support for supporting at least a part of the patient. This may allow to precisely position the arrangement of electrodes at a fixed position relative to the patient and/or relative to the body part.

In a seventh aspect, the present disclosure is directed to a medical system, wherein the system comprises the apparatus, as described above and in the following (e.g. the apparatus according to the sixth aspect), and a medical device for carrying out a medical procedure on the patient.

In an example of the system according to the seventh aspect, the medical device comprises a radiation treatment apparatus comprising a treatment beam source and a patient support unit (such as at least one of a patient bed or a headrest).

The present disclosure also relates to the use of any of the first to seventh aspect for determining a displacement of an internal object disposed partly or entirely in a body of a patient.

Moreover, it is emphasized that features, functions, elements and/or steps, which are described above and in the following with reference to one aspect of the invention, equally apply to any other aspect of the invention described above and in the following. Particularly, features and/or steps, as described above and in the following, with reference to the method according to the first aspect, equally apply to the computer program according to the second aspect, to the computer-readable medium according to the third aspect, to the computer according to the fourth aspect, to the use of the capacitive sensor according to the fifth aspect, to the apparatus according to the sixth aspect and/or to the medical system according to the seventh aspect, and vice versa.

These and other aspects of the disclosure will be apparent from and elucidated with reference to the embodiments described hereinafter.

Definitions

In this section, definitions for specific terminology used in this disclosure are offered which also form part of the present disclosure.

Computer Implemented Method

The method in accordance with the present disclosure is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

Acquiring Data

The expression “acquiring data” and/or “acquiring values” as used hereinabove and/or hereinbelow for example encompasses (within the framework of a computer implemented method) the scenario in which the data or values (e.g. capacitance values) are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data or values” also for example encompasses the scenario in which the data or values are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data or values” can therefore also for example mean waiting to receive data and/or receiving the data. The received data or values can for example be inputted via an interface. The expression “acquiring data or values” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data or values acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

Registering

The n-dimensional image of a body is registered when the spatial location of each point of an actual object within a space, for example a body part in an operating theatre, is assigned an image data point of an image (CT, MR, etc.) stored in a navigation system.

Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

Treatment Beam

The present invention relates to the field of controlling a treatment beam, beam and/or radiation beam. The treatment beam treats body parts which are to be treated and which are referred to in the following as “treatment body parts”. These body parts are for example parts of a patient's body, i.e. anatomical body parts.

The present invention relates to the field of medicine and for example to the use of beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

Reference is also made in this respect to the following web pages: http://www.elekta.com/healthcare_us_elekta_vmat.php and http://www.varian.com/us/oncology/treatments/treatment_techniques/rapidarc.

Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

Mapping

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 shows an apparatus for determining a displacement of an internal object according to an exemplary embodiment;

FIG. 2 shows a capacitive sensor of an apparatus for determining a displacement of an internal object according to an exemplary embodiment;

FIG. 3 shows a capacitive sensor of an apparatus for determining a displacement of an internal object according to an exemplary embodiment;

FIGS. 4A to 4C each show a capacitive sensor of an apparatus for determining a displacement of an internal object according to an exemplary embodiment;

FIG. 5 shows a flowchart illustrating steps of a method for determining a displacement of an internal object according to an exemplary embodiment; and

FIG. 6 shows a flowchart illustrating steps of a method for determining a displacement of an internal object according to an exemplary embodiment.

The figures are schematic only and not true to scale. In principle, identical or like parts, elements and/or steps are provided with identical or like reference numerals in the figures.

### DESCRIPTION OF EMBODIMENTS

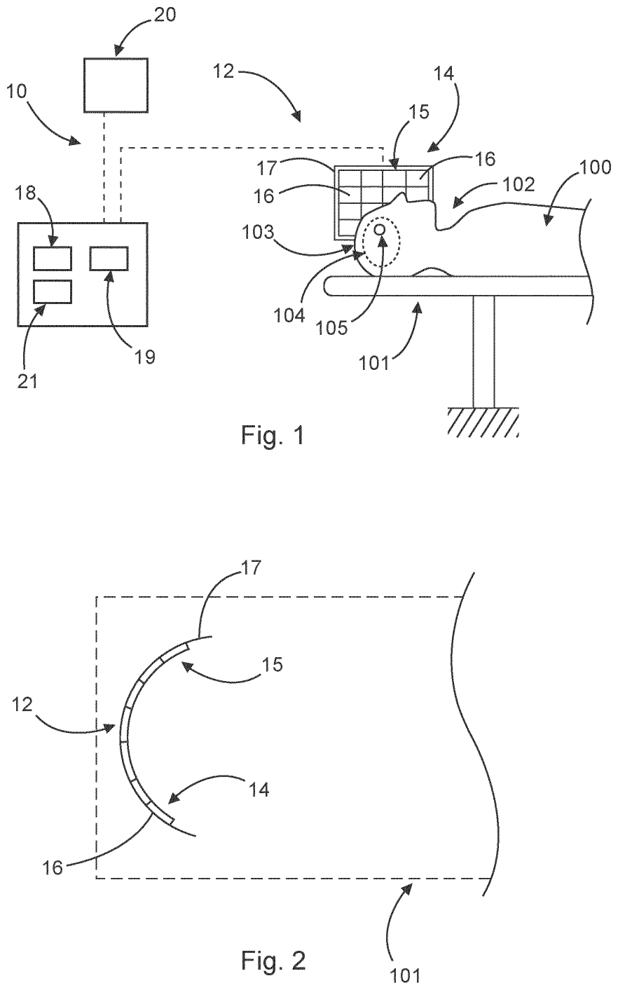

FIG. 1 shows an apparatus 10 for determining a displacement of an internal object 104 disposed in a body 102 of a patient 100 according to an exemplary embodiment in accordance with the sixth aspect of the present disclosure.

In the example shown in FIG. 1 the internal object 104 is a part of or constitutes the entire brain of the patient 100. Alternatively, the internal object 104 can be at least a part of an internal organ of the patient 100, at least a part of a lung of the patient 100, at least a part of a liver of the patient 100, at least a part of a spine of the patient 100 or any other internal structure or object of the patient. The internal object 104 is disposed and/or arranged in a body part 103 of the patient, which in the example shown in FIG. 1 refers to a head 103 of the patient 100.

A part 105 of the patient's brain is to be excised, removed and/or resected, e.g. in a surgical treatment or radiation treatment, wherein resection of part 105 can lead to a displacement and/or shift of the internal object 104 and/or a part of the patient's 100 brain.

For determining such displacement and/or shift, the apparatus 10 comprises a capacitive sensor 12 including at least one arrangement 14 of electrodes 16 having a plurality of electrodes 16. The arrangement 14 of electrodes 16 is positioned adjacent to, close to and/or near the body part 103 or head 103 of the patient 100, such that each electrode 16 of the arrangement 14 is spaced apart from the body part 103 or head 103 and/or such that each electrode 16 is not in contact with the patient 100.

In the example shown in FIG. 1, the arrangement 14 of electrodes 16 includes an array 15 and/or grid 15 of electrodes 16. Specifically, the arrangement 14 includes a grid 15 of sixteen electrodes 16 arranged in a two-dimensional configuration on a substrate in four lines and four rows. Any other dimension of the grid 15 or array 15 is conceivable. Also, other configurations, e.g. as described with reference to subsequent figures, or a plurality of arrangements 14 can be used.

The apparatus 10 further comprises a processor 18, a processing circuitry 18 and/or a control circuitry 18 configured to process sensor signals provided by the capacitive sensor 12.

Further, the apparatus 10 comprises a data storage 19 or data storage device, on which a set of reference capacitance values can be stored. These reference capacitance values can e.g. be determined before or during performing the actual medical treatment, as described in more detail hereinabove and hereinbelow.

For determining the displacement of the object 104, the capacitive sensor 12 is driven and generates a plurality of sensor signals, each being indicative of a capacitance in a vicinity of at least one electrode 16. Therein, the capacitance in the vicinity of the at least one electrode 16 of the arrangement of electrodes 12 is at least one of a capacitance between said at least one electrode 16 and a ground or ground potential, between said at least one electrode 16 and an outer surface of the patient 100, between said at least one electrode 16 and a reference electrode of the capacitance sensor 12, and between said at least one electrode 16 and a further electrode 16 of the capacitance sensor 12.

The processor 18 then receives and processes the plurality of sensor signals of the electrodes 16 to determine a set of capacitance values for at least a subset of the electrodes 16 of the arrangement 14 of electrodes 16. In other words, the processor 18 may acquire the set of capacitance values, e.g. at a measurement time.

The processor 18 then compares the set of determined capacitance values, e.g. elementwise and/or electrode-wise, with the reference capacitance values of the set of reference capacitance values. The reference capacitance values may have been determined and/or acquired at a reference time preceding the measurement time. For instance, the reference capacitance values may have been determined before resecting and/or irradiation the part 105 of the patient's 100 brain in a measurement, wherein the arrangement 14 is positioned at a reference position and/or in a reference orientation with respect to the body part 103 or head 103 of the patient 100. Further, the set of capacitance values may have been acquired and/or determined after the part 105 of the patient's 100 brain has been resected and/or irradiated. Due to this treatment, the internal object 104 and/or a part of the brain has been displaced with respect to and/or relative to the body part 103 or head 103. Accordingly, based on and/or by comparing the determined set of capacitance values with the set of reference capacitance values, this displacement of the internal object 104 can reliably be determined with high precision and accuracy. Also, markers attached to the patient 100 may not be required.

Generally, the processor 18 may determine an occurrence of the displacement, an extent or size of the displacement and/or a direction of the displacement. The direction can be determined in two or three spatial directions, e.g. if the arrangement 14 of electrodes 16 has a three-dimensional configuration as will be further discussed in subsequent figures.

It should be noted that the set of capacitance values can be determined or updated over time, e.g. during the medical treatment, thereby allowing to track a position and/or orientation of the internal object 104. Therein, “tracking” may mean determining over time.

Further, if the displacement is detected and/or determined, an alert may be provided, e.g. on user interface 21 of the apparatus 10, which may provide guidance to a surgeon or operator of the apparatus 10 as to whether a displacement of the object 104 has occurred and/or to which position and/or orientation the object 104 has moved.

In the example shown in FIG. 1, the arrangement 14 of electrodes 16 is arranged on and/or mounted to a patient support 101 supporting at least a part of the patient 100. Specifically, the arrangement 14 of FIG. 1 is mounted to an edge or rail of the patient support 101. Any other configuration is conceivable. For instance, the arrangement 14 may span or bridge at least partly over the patient support 101, such that the patient 100 is positionable between the arrangement 14 of electrodes 16 and the patient support 101. Also, the arrangement 14 of electrodes 16 may be arranged on top of the patient support 101, such that patient 100 can be positionable above the arrangement 14 of electrodes 16.

Further, the arrangement 14 of electrodes 16 could likewise be mounted on at least one of a head clamp, e.g. a Mayfield clamp, for immobilizing a head of the patient 100, an immobilization mask for immobilizing at least a part of the patient 100.

Moreover, the apparatus 10 comprises a surface scanner 20 and/or distance sensor 20. The surface scanner 20 and/or the distance sensor 20 can, for example, be a camera, a surface camera, a thermo-camera, a 3D camera, a stereo camera, a range camera, a laser sensor, a LIDAR sensor, a radar sensor, a time-of-flight sensor, or an ultrasound sensor.

Based on the surface scanner 20 and/or distance sensor 20, the a patient motion and/or global displacement of the object 104 can be determined in order to determine whether or not a displacement of the internal object 104 with respect to and/or relative to the body part 103 or head 103 has occurred. For instance, a first position of at least a part of a skin of the patient 100 may be determined at a first time with the surface scanner 20 and/or a first distance between at least a part of the patient 100 and at least one fixed point in an environment of the patient 100 may be determined with the distance sensor 20 at a first time. At a second time different than the first time, e.g. subsequent to the first time, a second position of the at least part of the skin and/or a second distance between the at least part of the patient 100 and the at least one fixed point in the environment of the patient 100 may be determined. By comparing the first and second position and/or the first and second distance, a patient motion and/or global displacement of the object 104 may be detected. This may allow reliably determining whether a displacement of the internal object 104 relative to the body part 103 (local displacement) or a displacement of the body part 103 relative to the arrangement 14 of electrodes 16 (global displacement) has occurred.

FIG. 2 shows a capacitive sensor 12 of an apparatus 10 for determining a displacement of an internal object 104 disposed in a body 102 of a patient 100 according to an exemplary embodiment in accordance with the sixth aspect of the present disclosure. If not stated otherwise, the capacitive sensor 12 of FIG. 2 comprises the same elements and/or features as the capacitive sensor 12 described with reference to FIG. 1.

In the example shown in FIG. 2, the arrangement 14 of electrodes 16 has a three-dimensional configuration. This may allow to infer three-dimensional information and/or information in three-dimensional space from the sensor signals and/or the determined set of capacitance values. Accordingly, the displacement of the internal object 104 may be determined in three-dimensional space.

Further, the electrodes 16 of the arrangement 14 of electrodes 16 are arranged in a semi-spherical and/or arc-like configuration, such that the arrangement 14 encompasses and/or surrounds at least a part of the patient's head 103 or body part 103.

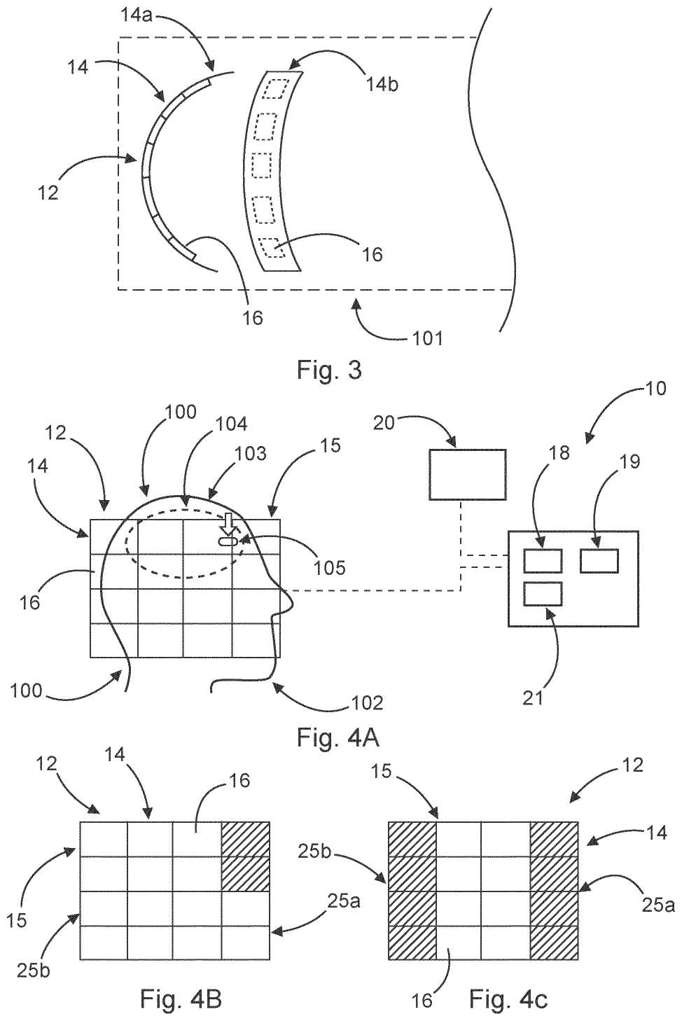

FIG. 3 shows a capacitive sensor 12 of an apparatus 10 for determining a displacement of an internal object 104 disposed in a body 102 of a patient 100 according to an exemplary embodiment in accordance with the sixth aspect of the present disclosure. If not stated otherwise, the capacitive sensor 12 of FIG. 3 comprises the same elements and/or features as the capacitive sensors 12 described with reference to FIGS. 1 and 2.

In the example shown in FIG. 3, the capacitive sensor 12 and/or the arrangement 14 of electrodes 16 includes an arc-shaped array 14a of electrodes 16 and a further arc-shaped array 14b of electrodes 16, wherein the arc-shaped array 14a and the further arc-shaped array 14b are directed and/or extend in different directions and/or spatial directions. Therein, each of arrays 14a, 14b encompasses and/or surrounds at least a part of the patient's head 103 or body part 103. Specifically, the array 14a at least partly encompasses a top of the patient's head 103 and the further array 14b at least partly encompasses a forehead of the patient 100. The further array 14b spans bridge-like across at least a part of the patient support 101.

FIG. 4A to 4C each show a capacitive sensor of an apparatus 10 for determining a displacement of an internal object 104 disposed in a body 102 of a patient 100 according to an exemplary embodiment in accordance with the sixth aspect of the present disclosure. Specifically, FIG. 4A shows the apparatus with capacitive sensor 12 and FIGS. 4B and 4C illustrate the capacitive sensor 12 of the apparatus 10. If not stated otherwise, the apparatus 10 and/or the capacitive sensors 12 of FIGS. 4A to 4C comprise the same elements and/or features as the capacitive sensors 12 described with reference to FIGS. 1 to 3.

The capacitive sensor 12 and/or the arrangement 14 of electrodes 16 of FIGS. 4A to 4C have a similar configuration as described with reference to FIG. 1. Further, FIG. 4B illustrates changes in the capacitance values or capacitances of the electrodes 16 that can occur when a local displacement of the internal object 104, i.e. a displacement of the object 104 relative to the body part 103 occurs (indicated by the arrow in FIG. 4A). FIG. 4C on the other hand illustrates changes in the capacitance values or capacitances of the electrodes 16 that can occur when a global displacement of the internal object 104, i.e. a displacement of the body part 103 relative to the arrangement 14 of electrodes 16 occurs.

The arrangement 14 of electrodes 16 in the example of FIGS. 4A to 4C is a grid 15 that is arranged close to the patient's 100 head 103 or body part, but not in contact therewith. The grid 15 comprises four by four electrodes 16 on a square arranged laterally in juxtaposition with and/or near a side of the head 103 of the patient 100.

By means of the apparatus 10 and/or the capacitive sensor 12, the reference capacitance value for each electrode 16 can be measured, e.g. by applying a voltage and integrating over the current or any other suitable technique to determine the capacitance values, when the arrangement 14 of electrodes 16 is positioned at a reference position and/or reference orientation with respect to the body part 103. The reference capacitance values can then be stored in the data storage 19, e.g. in a matrix and/or e.g. a single value for each electrode 16.

Optionally, it can be determined and/or monitored whether a patient motion occurs using the surface scanner 20 and/or distance sensor 20, as e.g. described with reference to FIG. 1.

Further, e.g. during the actual medical treatment, the capacitance values of the electrodes 16 and/or the set of capacitance values may be determined and/or measured, e.g. continuously.

By comparing the determined set of capacitance values with the stored set of reference capacitance values, it may be decided whether or not a local displacement of the internal object 104 relative to the body part 103 has occurred and/or whether a global displacement of the body part 103 relative to the arrangement 14 of electrodes 16 has occurred.

If a local displacement is detected, optionally, at least one of a size and a direction of the displacement can be calculated with the processor 18.

For example, e.g. in a “signature based” evaluation, a deviation and/or difference of each capacitance value of the set of capacitance values and the corresponding reference capacitance value of the set of reference capacitance values can be calculated. In other words, the element-wise and/or electrode-wise difference between the current capacitance values and the reference capacitance values can be calculated for some of or all of the electrodes 16.

The calculated deviations and/or differences can then be compared to threshold values to identify the electrodes 16 among the arrangement 14 of electrodes 16, in the vicinity of which the capacitance has changed. Accordingly, the “change” in capacitance can be evaluated based on determining if the differences and/or deviations reach, are below or are above the corresponding threshold value.

If only the capacitance in the vicinity of individual electrodes has changed, a local displacement of the object 104 relative to the body part 103 has occurred. This scenario is illustrated in FIG. 4B, wherein the shaded electrodes 16 illustrate the individual electrodes 16 for which the capacitance value has changed with respect to the corresponding reference capacitance value.

Optionally, occurrence of the local displacement can be checked and/or cross-checked by excluding that a patient motion and/or a global displacement has occurred, e.g. using an external sensor, such as the surface scanner 20 and/or distance sensor 20 of FIG. 1.

FIG. 4C illustrates the scenario, in which a global displacement of the object 104 has occurred, wherein the shaded electrodes 16 illustrate the electrodes 16 for which the capacitance value has changed with respect to the corresponding reference capacitance value.

To detect a global displacement, the processor 18 may, for example, determine the numbers of electrodes 16 whose capacitance has changed and evaluate the position of the identified electrodes on in the arrangement 14 of electrodes 16. If, as illustrated in the example of FIG. 4C, a first number of electrodes 16 arranged at a first boundary 25a or border 25a of the arrangement 14 of electrodes 16 and a second number electrodes 16 arranged at a second boundary 25b or border 25b of the arrangement 14 of electrodes 16 are substantially equal, a global displacement of the body part 103 with respect to the arrangement 14 of electrodes 16 might have occurred.

It should be noted that the set of reference capacitance values and/or the threshold values for the differences and/or deviations can be determined based on a measurement and/or based on a simulation calculation, e.g. in a “model based” evaluation. For instance, the correspondence between the displacement of the object 104 relative to the body part 103, e.g. a brain shift or tumor movement, may be simulated using e.g. an finite-element-model. The threshold values, typical signatures, typical sensor signals and/or changes in the capacitance values can then be derived from the model.

Apart from that, an artificial intelligence module, a classifier, a classificatory, and/or an artificial neural network may be used to determine the displacement of the object 104 relative to the body part 103 and/or the global displacement of the body part. For training, medical images, e.g. X-ray, CT, MRT, or the like, could be acquired from a subject or a phantom during surgery and the capacitance values can be measured and/or acquired as a function of time. The changes of capacity, optionally with the extent and/or direction and/or type of an organ movement, can then be labelled, e.g. manually, and used as input data for the training. E.g. a trained neural network can then be used to detect patterns of capacitance values and/or changes in capacitance values to determine a local and/or global displacement of the object 104.

Moreover, a phantom may be used to determine the set of reference capacitance values and/or the threshold values for the deviations. For instance, the capacitance values may be determined from a plurality of phantoms e.g. selectively filled with water, to determine the correspondence between the amount of water and/or air as well as the position relative to the patient 100 and the measured capacitance values or their changes.

The apparatus 10 according to the present disclosure may be advantageously used for cranial surgery. Therein, the arrangement 14 of electrodes 16 could be mounted to a head clamp used for cranial surgery. Alternatively or additionally, the arrangement 14 of electrodes 16 may be part of a couch 101, table 101 and/or patient support 101 used for radiotherapy or surgery, e.g. in the thorax or abdomen.

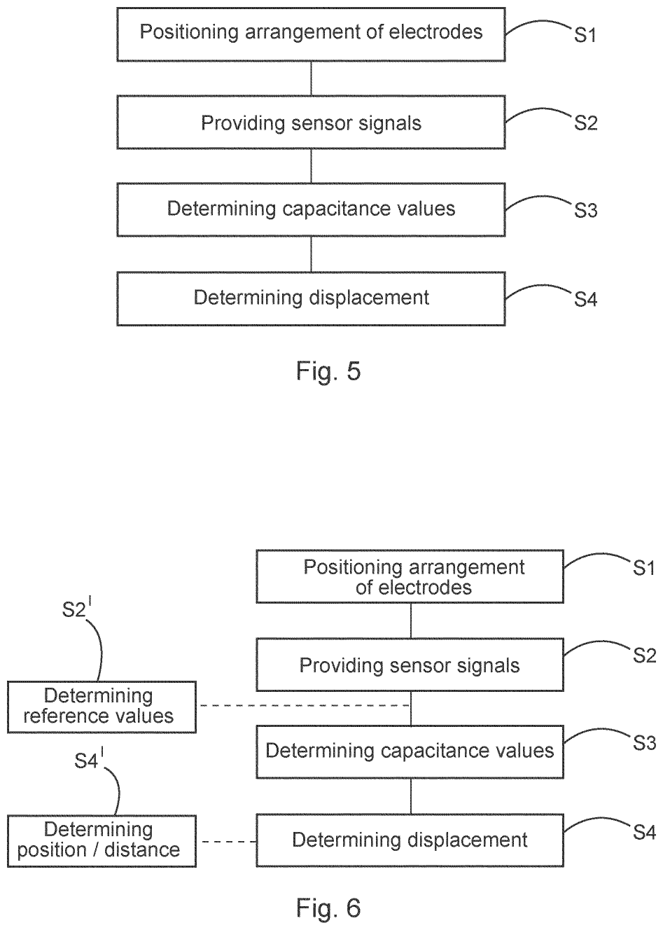

FIG. 5 shows a flowchart illustrating steps of a method for determining a displacement of an internal object 104 disposed in a body 102 of a patient 100 according to an exemplary embodiment in accordance with the method of the first aspect of the present disclosure. For performing the method, e.g. the apparatus 10 of the previous figures and/or parts thereof may be used.

In a first step S1, the arrangement 14 of electrodes 16 of the capacitive sensor 12 is arranged adjacent to the body part 103 of the patient 100, such that each electrode 16 is spaced apart from the body part 103.

In a second step S2, a plurality of sensor signals, is provided with the capacitive sensor 12, wherein each sensor signal is indicative of a capacitance in a vicinity of at least one electrode 16 of the arrangement 14 of electrodes 16.

In a further step S3, the set of capacitance values for at least a subset of the electrodes 16 of the arrangement 14 of electrodes 16 is determined with the processor 18 based on processing the plurality of sensor signals.

Further, the method comprises in step S4 determining the displacement of the internal object 104 with respect to the body part 103 based on comparing the determined set of capacitance values with the set of reference capacitance values, which may e.g. be stored in the data storage 19 or retrieved from another source.

Therein, step S4 may comprise determining the occurrence of the displacement of the internal object 104 with respect to the body part 103. Optionally, at least one of a size and a direction of the displacement of the internal object 104 with respect to the body part 103 may be calculated with the processor 18.

Moreover, determining the displacement of the internal object 104 relative to the body part 103 in step S4 may optionally comprise computing a deviation between each capacitance value of the determined set of capacitance values and at least one reference capacitance value of the set of reference capacitance values, and comparing the computed deviation for each capacitance value of the determined set of capacitance values with a threshold value, thereby determining whether the capacitance in the vicinity of one or more electrodes 16 has changed.

Further, it may be determined whether the capacitance in the vicinity of one or more individual electrodes 16 has changed in order to detect a local displacement, as described hereinabove.

Alternatively or additionally, it may be checked in step S4 whether a patient motion relative to the arrangement 14 of electrodes 16 has occurred, e.g. based on determining the numbers of electrodes 16 and/or their position in the arrangement 14 of electrodes 16 to determine a global displacement of the object 104. For instance, a first number of electrodes 16 arranged at the first boundary 25a of the arrangement 14 of electrodes 16, in the vicinity of which the capacitance has changed, and a second number of electrodes 16 arranged at the second boundary 25b of the arrangement 14 of electrodes 16, in the vicinity of which the capacitance has changed, may be compared to each other, as described hereinabove. If the first and second number substantially match, a global displacement might have occurred.

FIG. 6 shows a flowchart illustrating steps of a method for determining a displacement of an internal object 104 disposed in a body 102 of a patient 100 according to an exemplary embodiment in accordance with the method of the first aspect of the present disclosure. For performing the method, e.g. the apparatus 10 of the previous figures and/or parts thereof may be used. If not stated otherwise, the method of FIG. 6 comprises the same steps as the method of FIG. 5.

The method of FIG. 6 further comprises optional step S2′, in which the set of reference capacitance values is determined based on positioning the arrangement 14 of electrodes 16 at a reference position and in a reference orientation with respect to the body part 103 of the patient 100 and based on measuring the reference capacitance values using the capacitive sensor 12, e.g. on the patient 101 or using a phantom. Alternatively or additionally, the set of reference capacitance values can be determined based on a simulation calculation with the arrangement 14 of electrodes 16 being positioned at the reference position and in the reference orientation with respect to the body part 103 of the patient 100. Also, the set of reference capacitance values may be stored in the data storage 19 in step S2′.

Further, the threshold values for the deviations between the capacitance values and the reference capacitance values may be determined in step S2′ based on a calibration measurement and/or based on a simulation calculation, as described hereinabove.

Moreover, FIG. 6 illustrates the optional step S4′, in which it is monitored and/or determined whether a global displacement of the object 104 has occurred, e.g. using the surface scanner 20 and/or the distance sensor 20, as described hereinabove.

For instance, a first position of at least a part of a skin of the patient 100 can be determined with the surface scanner 20 at a first time. Further, a second position of the at least part of the skin can be determined with the surface scanner 20 at a second time different than the first time, and a displacement of the internal object 104 with respect to the body part 103 can be determined based on comparing the determined first position and the determined second position of the at least part of the skin. This may mean that a local displacement can be determined based on detecting with the surface scanner 20 that no global displacement has occurred and/or based on excluding a global displacement using the surface scanner 20.

Alternatively or additionally, a first distance between at least a part of the patient 100 and at least one fixed point in an environment of the patient 100 can be determined with the distance sensor 20 at a first time. Further, a second distance between the at least part of the patient 100 and the at least one fixed point in the environment of the patient 100 can be determined with the distance sensor 20 at a second time different than the first time, and a displacement of the internal object 104 with respect to the body part 103 can be determined based on comparing the determined first distance and the determined second distance between the at least part of the patient 100 and the at least one fixed point. This may mean that a local displacement can be determined based on detecting with the distance sensor 20 that no global displacement has occurred and/or based on excluding a global displacement using the distance sensor 20.

It should be noted that in a further optional step, an alert signal can be generated in response to determining the displacement of the internal object 104 with respect to the body part 103.

Moreover, a pre-operative scan or image of at least a part of the body part 103 and the internal object 104 may be provided, and at least one of a shape, a geometry, a position and an orientation of the at least part of the internal object 104 may be adjusted in the pre-operative scan based on the determined displacement of the internal object 104 with respect to the body part 103.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope

1. A method for determining a displacement of an internal object disposed in a body of a patient, the method comprising:
positioning an arrangement of electrodes of a capacitive sensor adjacent to a body part of the patient, such that each electrode is spaced apart from the body part, wherein the body part at least partly encloses the internal object of the patient;
providing, with the capacitive sensor, a plurality of sensor signals, wherein each sensor signal is indicative of a capacitance in a vicinity of at least one electrode of the arrangement of electrodes;
determining a set of capacitance values for at least a subset of the electrodes of the arrangement of electrodes based on processing the plurality of sensor signals; and
determining a displacement of the internal object with respect to the body part based on comparing the determined set of capacitance values with a set of reference capacitance values.

2. The method according to claim 1, wherein determining the displacement of the internal object comprises:
determining an occurrence of the displacement of the internal object with respect to the body part; and
determining at least one of a size and a direction of the displacement of the internal object with respect to the body part.

3. The method according to claim 1, wherein the set of reference capacitance values is indicative of the capacitance values of the at least subset of electrodes when the arrangement of electrodes is at least one of positioned at a reference position or positioned in a reference orientation with respect to the body part.

4. The method according to claim 1, wherein at least one of the set of capacitance values is determined at a measurement time or the set of reference capacitance values is indicative of the capacitance values of the at least subset of electrodes at a reference time preceding the measurement time.

5. The method according to claim 1, further comprising one or more of:
at least one of each sensor signal or each capacitance value is indicative of at least one of a capacitance between one of the electrodes and a ground, between one of the electrodes and an outer surface of the patient, between one of the electrodes and a reference electrode of the capacitance sensor, or between two electrodes of the capacitance sensor; and
the capacitance in the vicinity of the at least one electrode of the arrangement of electrodes is at least one of a capacitance between said at least one electrode and a ground, between said at least one electrode and an outer surface of the patient, between said at least one electrode and a reference electrode of the capacitance sensor, and between said at least one electrode and a further electrode of the capacitance sensor.

6. The method according to claim 1, further comprising:
determining the set of reference capacitance values based on positioning the arrangement of electrodes at a reference position and in a reference orientation with respect to the body part of the patient and based on measuring the reference capacitance values using the capacitive sensor.

7. The method according to claim 1, further comprising:
determining the set of reference capacitance values based on a simulation calculation with the arrangement of electrodes being positioned at a reference position and in a reference orientation with respect to the body part of the patient.

8. The method according to claim 1, further comprising one or more of:
determining the set of reference capacitance values based on positioning the arrangement of electrodes at a reference position and in a reference orientation with respect to the body part of the patient and based on measuring the reference capacitance values using the capacitive sensor, and storing the determined set of reference capacitance values in a data storage; and
determining the set of reference capacitance values based on a simulation calculation with the arrangement of electrodes being positioned at a reference position and in a reference orientation with respect to the body part of the patient, and storing the determined set of reference capacitance values in a data storage.

9. The method according to claim 1, wherein determining the displacement of the internal object comprises:
computing a deviation between each capacitance value of the determined set of capacitance values and at least one reference capacitance value of the set of reference capacitance values; and
comparing the computed deviation for each capacitance value of the determined set of capacitance values with a threshold value, thereby determining whether the capacitance in the vicinity of one or more electrodes has changed.

10. The method according to claim 9, further comprising;
identifying one or more electrodes in the vicinity of which the capacitance has changed; and comprising one or more of:
determining an occurrence of the displacement of the internal object with respect to the body part based on determining that the capacitance in the vicinity of one or more individual electrodes has changed; and
at least one of checking or determining an occurrence of a patient motion relative to the arrangement of electrodes based on determining that the capacitance in the vicinity of at least one electrode arranged at a first boundary of the arrangement of electrodes and in the vicinity of at least one further electrode arranged at a second boundary opposite to the first boundary of the arrangement of electrodes has changed.

11. The method according to claim 10, wherein the occurrence of the patient motion relative to the arrangement of electrodes is at least one of checked or determined based on:
determining a first number of electrodes arranged at the first boundary of the arrangement of electrodes, in the vicinity of which the capacitance has changed;
determining a second number of electrodes arranged at the second boundary of the arrangement of electrodes, in the vicinity of which the capacitance has changed; and
comparing the first number of electrodes arranged at the first boundary with the second number of electrodes arranged at the second boundary of the arrangement of electrodes.

12. The method according to claim 11, wherein the occurrence of the patient motion relative to the arrangement of electrodes is at least one of checked or determined based on determining that the first number of electrodes arranged at the first boundary substantially equals or matches the second number of electrodes arranged at the second boundary of the arrangement of electrodes.

13. The method according to claim 9, wherein the threshold values for the deviations between the capacitance values and the reference capacitance values are determined based on at least one of a calibration measurement or a simulation calculation.

14. The method according to claim 1, wherein the displacement of the internal object with respect to the body part is determined using at least one of a classificator, an artificial intelligence module or a neural network.

15. The method according to claim 1, further comprising:
determining, with at least one surface scanner, a first position of at least a part of a skin of the patient at a first time;
determining, with the at least one surface scanner, a second position of the at least part of the skin at a second time different than the first time; and
determining an occurrence of the displacement of the internal object with respect to the body part based on comparing the determined first position and the determined second position of the at least part of the skin.

16. The method according to claim 1, further comprising:
determining, with at least one distance sensor, a first distance between at least a part of the patient and at least one fixed point in an environment of the patient at a first time;
determining, with the at least one distance sensor, a second distance between the at least part of the patient and the at least one fixed point in the environment of the patient at a second time different than the first time; and
determining an occurrence of the displacement of the internal object with respect to the body part based on comparing the determined first distance and the determined second distance between the at least part of the patient and the at least one fixed point.

17. The method according to claim 1, further comprising one or more of:
(i) determining, with at least one surface scanner, a first position of at least a part of a skin of the patient at a first time: determining, with the at least one surface scanner, a second position of the at least part of the skin at a second time different than the first time; and determining an occurrence of the displacement of the internal object with respect to the body part based on comparing the determined first position and the determined second position of the at least part of the skin; and
(ii) determining, with at least one distance sensor (20), a first distance between at least a part of the patient and at least one fixed point in an environment of the patient at a first time;
determining, with the at least one distance sensor (20), a second distance between the at least part of the patient and the at least one fixed point in the environment of the patient at a second time different than the first time; and determining an occurrence of the displacement of the internal object with respect to the body part based on comparing the determined first distance and the determined second distance between the at least part of the patient and the at least one fixed point;
wherein at least one of the at least one surface scanner or the at least one distance sensor is at least one of a camera, a surface camera, a thermo-camera, a 3D camera, a stereo camera, a range camera, a laser sensor, a LIDAR sensor, a radar sensor, a time-of-flight sensor, or an ultrasound sensor.

18. The method according to claim 1, further comprising:
generating an alert signal in response to determining the displacement of the internal object with respect to the body part.

19. The method according to claim 1, further comprising:
providing a pre-operative scan of at least a part of the body part, the pre-operative scan including at least a part of the internal object; and
adjusting at least one of a shape, a geometry, a position, or an orientation of the at least part of the internal object in the pre-operative scan based on the determined displacement of the internal object with respect to the body part.

20. The method according to claim 1, wherein the internal object is at least one of at least a part of an internal organ of the patient, at least a part of a brain of the patient, at least a part of a lung of the patient, at least a part of a liver of the patient, or at least a part of a spine of the patient.

21. (canceled)

22. An apparatus for determining a displacement of an internal object disposed in a body of a patient, the apparatus comprising:
a capacitive sensor including at least one arrangement of electrodes, wherein the at least one arrangement of electrodes is configured to be arranged adjacent to a body part of a patient, which body part at least partly encloses the internal object, and wherein the capacitive sensor is configured to generate a plurality of sensor signals, each sensor signal being indicative of a capacitance in a vicinity of at least one electrode of the arrangement of electrodes; and
at least one processor configured to receive and process the plurality of sensor signals to determine a set of capacitance values for at least a subset of the electrodes of the arrangement of electrodes;
wherein the at least one processor is configured to determine a displacement of the internal object with respect to the body part based on comparing the determined set of capacitance values with a set of reference capacitance values.

23. The apparatus according to claim 22, wherein the arrangement of electrodes includes at least one of an array of electrodes or a grid of electrodes.

24. The apparatus according to claim 22, wherein the electrodes of the arrangement electrodes are at least one of arranged in a uniform pattern, arranged in a three-dimensional configuration, or arranged in at least one of a semi-spherical configuration, a spherical configuration, or an arc-shaped configuration.

25. (canceled)

26. (canceled)

27. The apparatus according to claim 22, wherein the arrangement of electrodes includes an arc-shaped array of electrodes and at least one further arc-shaped array of electrodes; and wherein the arc-shaped array and the at least one further arc-shaped array are at least one of directed in different directions or extend in different directions.

28. The apparatus according to claim 22, wherein the electrodes of the arrangement of electrodes are arranged on at least one of a head clamp for immobilizing a head of the patient, an immobilization mask for immobilizing at least a part of the patient, and a patient support for supporting at least a part of the patient.
